(19) United States
(12) Patent Application Publication
(10) Pub. No.: US 2007/0239019 A1
(43) Pub. Date: Oct. 11, 2007

(54) Portable ultrasonic imaging probe than connects directly to a host computer

(76) Inventors: William D. Richard, Ballwin, MO (US); Roman Solek, Pleasanton, CA (US); David M. Zar, Maryland Heights, MO (US)

Correspondence Address: FLIESLER MEYER LLP, 650 CALIFORNIA STREET, 14TH FLOOR, SAN FRANCISCO, CA, 94108, US

(21) Appl. No.: 11/353,185

(22) Filed: Feb. 13, 2006

(51) Int. Cl. A61B 8/00

(52) U.S. Cl. 600/459; 600/443

(57) ABSTRACT

A portable ultrasonic imaging probe is adapted to connect to a host computer via a passive interface cable, e.g., a standard USB 2.0 peripheral interface cable or a standard IEEE 1394 “Firewire” peripheral interface cable. In accordance with an embodiment, the portable ultrasound imaging probe includes a probe head, a logarithmic compressor, an envelope detector, and analog-to-digital converter and interface circuitry, all of which receive power from the host computer via the passive interface cable. To simplify the portable ultrasonic imaging probe, none of electronic beamforming, time gain compensation, gray-scale mapping and scan conversion are performed within the probe. This abstract is not intended to describe all of the various embodiments of the present invention, or to limit the scope of the invention.

### FIELD OF THE INVENTION

The present invention relates to portable ultrasonic imaging probes, and more specifically, to such probes that can be directly connected to a host computer, such as an off-the-shelf laptop computer, or the like.

### BACKGROUND

Typically, ultrasound imaging systems include a hand-held probe that is connected by a cable to a relatively large and expensive piece of hardware that is dedicated to performing ultrasound signal processing and displaying ultrasound images. Such systems, because of their high cost, are typically only available in hospitals or in the offices of specialists, such as radiologists.

Recently, there has been an interest in developing more portable ultrasound imaging systems that can be used with personal computers. One such system, described in U.S. Pat. No. 6,440,071, includes an electronic apparatus that is connected between a personal computer and an ultrasound probe. The electronic apparatus sends and receives signals to and from an ultrasound probe, performs ultrasound signal processing, and then sends ultrasound video to a personal computer that displays the ultrasound video. A disadvantage of the system of the '071 patent is that there is a need for a custom electronic apparatus located between the probe and the personal computer. A further disadvantage of the system of the '071 patent is that analog signals travel a relatively long distance between the probe and the electronic apparatus, which will result in a poor signal-to-noise ratio. Another disadvantage of the system of the '071 patent is that the cable that carries analog signals between the probe and the electronic apparatus is a custom cable.

Another ultrasound imaging system that that can be used with personal computers is described in U.S. Pat. No. 6,969,352. This system includes an integrated front end probe that interfaces with a host computer, such as a personal computer. The integrated front end probe performs electronic beamforming and other signal processing, such as time gain compensation (TGC), using hardware that is dedicated to such finctions, and sends ultrasound video to the host computer that displays the ultrasound video. A disadvantage of the system of the '352 patent is that the components necessary to perform electronic beamforming as well as the components necessary to perform TGC within the integrated front end probe are relatively expensive. Another disadvantage is of the system of the '352 patent is that a custom cable, which includes a DC-DC converter, is used to connect the probe to the host computer.

Accordingly, there is still a need for an inexpensive portable ultrasound probe that can be used with an off-the-shelf host computer, such as a personal computer. Preferably, such a portable ultrasound probe is inexpensive enough to provide ultrasound imaging capabilities to general practitioners and health clinics having limited financial resources.

### SUMMARY

Embodiments of the present invention relate to a portable ultrasonic imaging probe that is adapted to connect to a host computer via a passive interface cable, such us, but not limited to, a standard USB 2.0 peripheral interface cable or a standard IEEE 1394 “Firewire” peripheral interface cable.

In accordance with an embodiment, the portable ultrasound imaging probe includes a probe head, a logarithmic compressor, an envelope detector, and analog-to-digital converter and interface circuitry. The probe head includes a maneuverable single-element transducer to send ultrasonic pulses and detect ultrasonic echoes. The logarithmic compressor performs logarithmical compression of analog echo signals representative of the detected ultrasonic echoes. The envelope detector performs envelope detection of the logarithmically compressed analog echo signals. The analog-to-digital converter converts the logarithmically compressed and envelope detected analog echo signals to digital signals representative of the logarithmically compressed and envelope detected echo signals. The interface circuitry transfers the digital signals representative of the logarithmically compressed and envelope detected echo signals across the passive interface cable to a host computer, so that the host computer can perform time gain compensation, gray-scale mapping and scan conversion of the data, and display ultrasound images on a display associated with the host computer.

In accordance with an embodiment, the logarithmic compressor and the envelope detector are collectively embodied in a logarithmic amplifier. In other words, the logarithmic amplifier receives the analog echo signals representative of the detected ultrasonic echoes, performs both logarithmic compression and envelope detection of the analog echo signals, and outputs the logarithmically compressed and envelope detected analog echo signals.

In accordance with embodiments of the present invention, in order to provide for a relatively simple and inexpensive portable ultrasound imaging probe, the portable ultrasound imaging probe does not perform any of time gain compensation, gray-scale mapping and scan conversion. Rather, these functions are performed within the host computer that receives the digital data from the portable probe. Also, because the probe head includes a maneuverable single-element transducer, there is no need for the portable ultrasound imaging probe, or the host computer for that matter, to perform any electronic beamforming.

In accordance with embodiments of the present invention, the probe head assembly, the logarithmic compressor, the envelope detector, the analog-to-digital converter and the interface circuitry all receive power from the host computer via the same passive interface cable across which the probe transfers the digital signals to the host computer. This can be accomplished by including voltage regulator circuitry, within the portable ultrasonic imaging probe, to receive a power signal from the host computer via the passive interface cable, and to produce voltages used to power the aforementioned components.

Additionally, the probe head assembly includes a pulser to provides high voltage pulses to the transducer to cause the transducer to send ultrasonic pulses. In accordance with an embodiment of the present invention, power for the pulser is received from a high voltage power supply within the portable ultrasonic imaging probe, where the high voltage power supply steps-up a voltage of the power signal, received from the host computer via the passive interface cable, to thereby produce the higher voltage that powers the pulser.

The portable ultrasound imaging probe may also include a pre-amplifier and a filter, wherein the analog echo signals are preamplified and filtered by the pre-amplifier and the filter before being provided to the logarithmic compressor.

This description is not intended to be a complete description of, or limit the scope of, the invention. Alternative and additional features, aspects, and objects of the invention can be obtained from a review of the specification, the figures, and the claims.

### BRIEF DESCRIPTION OF THE DRAWINGS

FIG. 1A is a high level diagram that is useful for describing embodiments of the present invention.

FIG. 1B illustrates a specific implementation of the invention originally described with reference to FIG. 1A.

FIG. 2 is a block diagram that shows additional details of an ultrasonic imaging probe according to an embodiment of the present invention.

FIG. 3 illustrates additional details of the buck regulator (BUCK REG) shown in FIG. 2, according to a specific embodiment of the present invention.

FIG. 4 illustrates additional details of the high voltage power supply (HVPS) shown in FIG. 2, according to a specific embodiment of the present invention.

### DETAILED DESCRIPTION

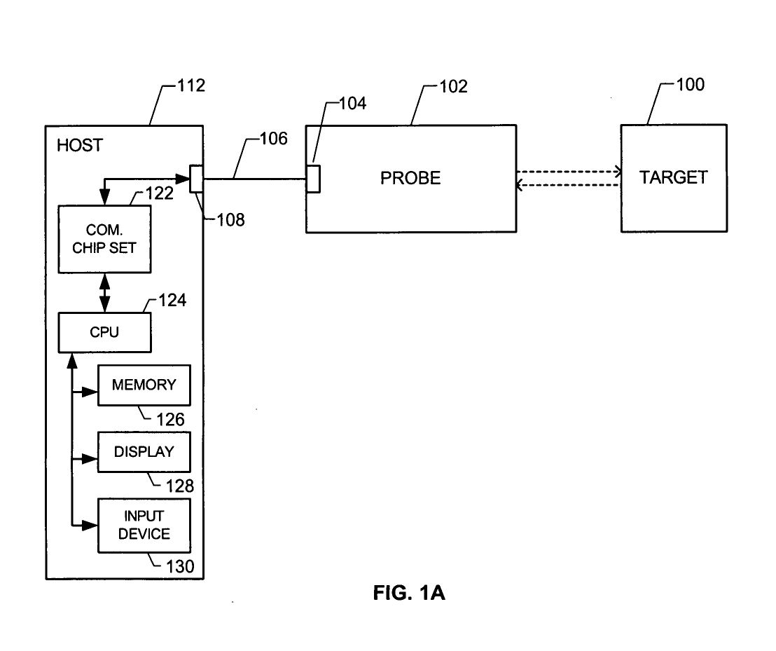

FIG. 1A shows an ultrasonic imaging probe 102, according to an embodiment of the present invention, that is connected by a passive interface cable 106 to a host computer 112. The host computer 112 can be a desktop personal computer (PC), a laptop PC, a pocket PC, a tablet PC, a cell phone capable or running software programs (e.g., a Palm Treo™), a personal digital assistant (e.g., a Palm Pilot™), or the like. The passive interface cable 106, which includes connectors and passive wires, can be a Universal Serial Bus (USB) cable (e.g., a USB 2.0 cable), a FireWire (also known as IEEE 1394) cable, or the like. Preferably the probe 102 is not connected to any other device or power supply. Thus, as will be described below, in a preferred embodiment the probe 102 receives all its necessary power from the host computer 112 via the passive interface cable 106.

As will be described in more detail below, in accordance with embodiments of the present invention, the probe 102 enables the host computer 112, via software running on the host computer 112, to form real-time ultrasonic images of a target 100 (e.g., human tissue or other materials) without the need for any additional internal or external electronics, power supply, or support devices. More specifically, the probe 102 produces raw digitized data that is logarithmically compressed, envelope detected ultrasound echo data from a single transducer in the probe 102, and transmits such raw data to the host computer 112. When the host computer 112 receives raw data via the passive interface cable 106 from the probe 102, the host computer 112 performs time gain compensation (TGC), gray-scale mapping, and scan conversion of the raw data using software that runs on the host computer 112, and displays the resultant video images. No electronic beamforming or other equivalent image processing is implemented by the probe 102, thereby reducing the complexity and cost of the probe 102. Additionally, because a single maneuverable transducer is used to obtain the raw ultrasound data, there is no need for any electronic beamforming or other equivalent image processing to be performed on the data once it is transferred to the host computer 112, thereby simplifying the software that the host computer 112 runs, and thus reducing the required processing capabilities of the host computer 112. The term “raw data”, as used herein, refers to ultrasound imaging data that has not yet been time gain compensated, gray-scale mapped and scan converted. As described below, such raw data is included in the digital signals that are transferred from the probe 102 to the host computer 112.

As shown in FIG. 1A, the host computer 112 will likely include a communications port 108, a communications chip-set 122, a central processing unit (CPU) 124, memory 126, a display 128, and an input device 130, such as a keyboard, mouse, touch screen, track ball, or the like. Additionally, the host computer 112 runs software that enables the host to control specific aspects of the probe 102. Such software also enables the host computer 112 to perform time gain compensation (also known as time gain correction), gray-scale mapping, and scan conversion of the raw data received from the probe 112 over the passive interface cable 106. The host computer 112 can then display the resulting ultrasound video on the display 128, as well as store such video in its memory 126, or another data storage device (not shown). The article “A New Time-Gain Correction Method for Standard B-Mode Ultrasound Imaging”, by William D. Richard, IEEE Transactions of Medical Imaging, Vol. 8, No. 3, pp. 283-285, September 1989, which is incorporated herein by reference, describes an exemplary time gain correction technique that can be performed by the host computer 112. The article “Real-Time Ultrasonic Scan Conversation via Linear Interpolation of Oversampled Vectors,” Ultrasonic Imaging, Vol. 16, pp. 109-123, April 1994, which is incorporated herein by reference, describes an exemplary scan conversion technique that can be performed by the host computer 112.

The passive interface cable 106 includes at least one data line over which data is carried, and at least one power line to provide power to a peripheral device, which in this case is the ultrasonic imaging probe 102. For example, where the passive interface cable 106 is a USB 2.0 cable, one wire of the cable provides about 5V at about ½ Amp. In alternative embodiments, the passive interface cable 106 is a Firewire cable, which also includes a power wire. Other types of passive interface cable can be used if desired. However, as mentioned above, it is preferred that the passive interface cable 106 is a standard off-the-shelf cable that can interface with an off-the-shelf interface IC. The term passive as used herein refers to a cable that does not regenerate signals or process them in any way.

FIG. 1B illustrates an example where the host computer 112 is a laptop. FIG. 1B also shows an exemplary ergonomic design of a housing 103 for the ultrasonic imaging probe 102 of the present invention. Other ergonomic designs are of course possible, and within the scope of the present invention. Also, as explained above, other types of host computer 112 can also be used.

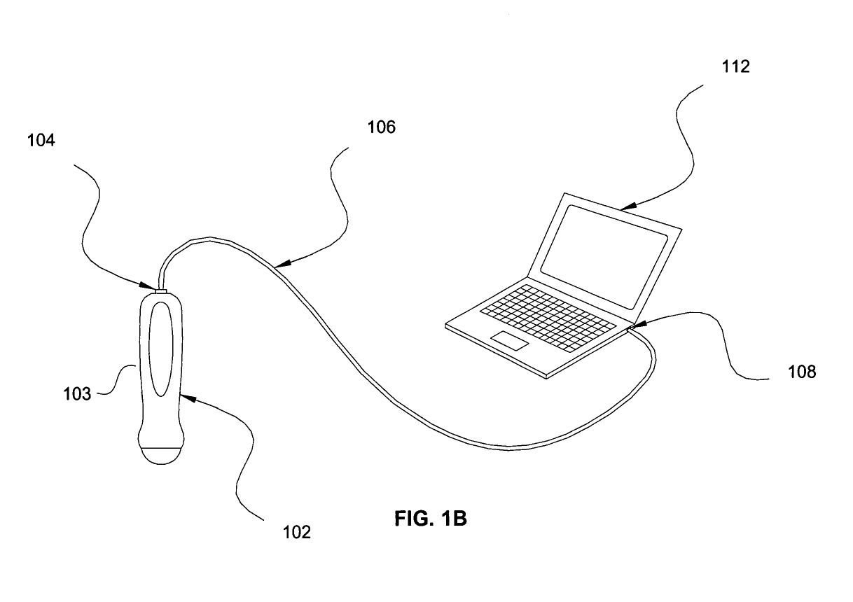

Additional details of the ultrasonic imaging probe 102, according to specific embodiments of the present invention, are shown in FIG. 2. As shown in FIG. 2, in accordance with an embodiment of the present invention, the probe 102 includes a peripheral connector 104 and an interface IC 204 that enables the probe 102 to interface with the host computer 112 via the interface cable 106. The connector 104 and the interface IC 204 are preferably off-the-shelf devices, but can be custom devices. In one embodiment, the connector 104 is a FireWire connector, and the interface IC 204 is a FireWire interface IC. In another embodiment, the connector 104 is a Universal Serial Bus (USB) connector, and the interface IC 204 is a USB interface IC. An exemplary off-the-shelf IC that can be used to implement a USB interface is the CY7C8014A EZ-USB FX2LP™ USB Microcontroller available from Cypress Semiconductor Corp. of San Jose, Calif., which integrates a USB 2.0 interface, 4 KB of static random access memory (SRAM) for buffering high-speed USB data, and an 8051 microprocessor with 16 KB of code/data SRAM all integrated into a single chip. This chip can run embedded 8051 code that is stored in a serial programmable read only memory (SPROM) 246 that is accessible via an internal bus 244 (e.g., an Inter-Integrated Circuit (I2C) bus) or that has been downloaded from the host computer 112 via a process called ReNumeration, which is discussed in Cypress Semiconductor Corporation's “EZ-USB FX2LP™ USB Microcontroller Datasheet,” Cypress Document Number 38-8032 Rev I, Jun. 1, 2005, which is incorporated herein by reference.

In accordance with an embodiment of the present invention, the portable ultrasound imaging probe 102 includes a single transducer 270 that is pivoted by a shaft 254 that is connected to a motor 250. An encoder 252, which can be mechanical, optical, or some other type, is used to provide feedback indicative of the position of the motor shaft 254 (and thus the position of the transducer 270) to the microcontroller of the interface IC 204 and to a programmable logic device or programmable gate array, which in the embodiment shown is a complex programmable logic device (CPLD) 206. As shown in FIG. 2, the transducer 270, the motor 250, the encoder 252 and the shaft 254 are components of the probe head assembly 280. In one embodiment, the position of the transducer is represented by an one byte of data, such that there can be 256 different positions of the transducer 270 (i.e., position 0 through position 255).

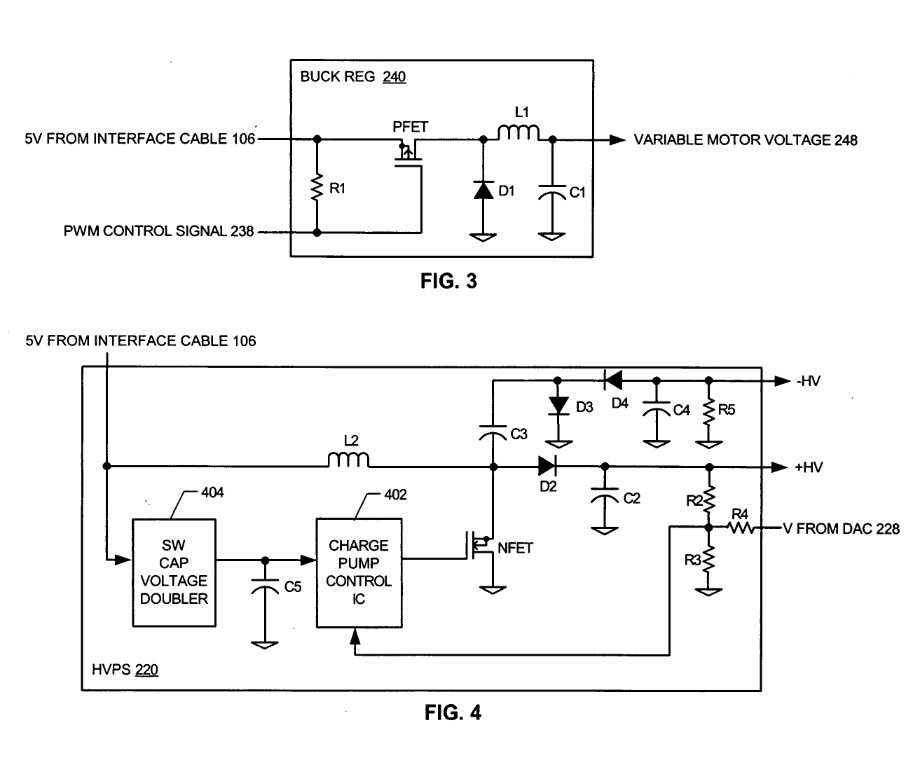

The ultrasonic imaging probe 102 includes an ultrasonic pulser 208 that sends precisely timed drive pulses to the transducer 270, through the transmit/receive (T/R) switch 210, to initiate transmission of ultrasonic pulses. The pulser 208 is configured to provide pulses that are sufficient to drive the transducer 210 to ultrasound oscillation. The host computer 112, through the passive interface cable 106, the interface IC 204 and the CPLD 206, can control the amplitude, frequency and duration of the pulses output by the pulser 208 via the pulse control line 207. The pulser 208 is powered by a high voltage power supply (HVPS) 220, which generates the necessary high voltage potential required by the pulser 208 from a lower voltage (e.g., 5V) received via the passive interface cable 106. Additional details of the HVPS 220, according to an embodiment of the present invention, are discussed below with reference to FIG. 4.

The pulser 208 is preferably a bi-polar pulser that produces both positive and negative high voltage pulses that can be as large as +/−100V. In such an embodiment, the HVPS 220 provides up to +/−100V supply rails to the pulser 208. A digital-to-analog converter (DAC) 228 that is connected to the internal bus 244 is used to set the peak voltage produced by the HVPS 220. In a specific embodiment, the commands used to control the bus 228 are generated by the microprocessor (e.g., an 8051 microprocessor) of the interface IC 204. An exemplary IC that can be used to implement the bus 228 is the AD5301 Buffered Voltage Output 8-Bit DAC available from Analog Devices of Norwood, Mass. Additional details of the HVPS 220, according to an embodiment of the present invention, are described below with reference to FIG. 4.

The T/R switch 210 is used to connect the switch 270 to either the pulser 208 or a pre-amplifier 212. When a high voltage pulse is produced by the pulser 208, the T/R switch 210 automatically blocks the high voltage from damaging the pre-amplifier 212 while delivering the pulse to the switch 270 via a pulse path 272, which can be, e.g., a short 50 ohm coaxial line. When the pulser 208 is not producing a pulse, the T/R switch 210 automatically switches to disconnect the switch 270 from the pulser 208, and to connect the switch 270 (via the pulse path 272) to the pre-amplifier 212.

The transducer 270, e.g., a piezoelectric element, transmits ultrasonic pulses into the target region being examined and receives reflected ultrasonic pulses (i.e., “echo pulses”) returning from the region. As described above, the T/R switch 220 enables the probe 102 to alternate between transmitting and receiving. When transmitting, the transducer 270, is excited to high-frequency oscillation by the pulses emitted by the pulser 208, thereby generating ultrasound pulses that can be directed at a target region/object to be imaged. These ultrasound pulses (also referred to as ultrasonic pulses) produced by the switch 270 are echoed back towards the switch 270 from some point within the target region/object, e.g., at boundary layers between two media with differing acoustic impedances. Then, when receiving, the “echo pulse” is received by the switch 270 and converted into a corresponding low-level electrical input signal (i.e., the “echo signal”) that is provided to the pre-amplifier 212 for enhancing the signal.

The pre-amplified echo signal output by the pre-amplifier 212 is provided to a filter, such as a low pass filter (LPF) 214 or a bandpass filter, which filters out the frequencies that are not of interest. The pre-amplifier 212, in accordance with an embodiment, is a very low noise amplifier that provides about 20 dB of gain. The LPF 214, in accordance with an embodiment, is a passive, four-pole, band limited low pass filter.

The filtered pre-amplified echo signal output by the filter 214, which is a radio frequency (RF) signal, is provided to a logarithmic amplifier 216. The logarithmic amplifier 216 performs log-compression and envelope detection of the filtered pre-amplified echo signal, thereby compressing the dynamic range of the echo signal. An exemplary finction of the logarithmic amplifier 216 can be

$${V}_{OUT} = {V}_{Y} log ( \frac{{V}_{IN}}{{V}_{X}} ) ,$$

where V_{OUT} is the voltage output by the logarithmic amplifier 216, V_{y} is the slope voltage, V_{IN} is the voltage input to the logarithmic amplifier 216 (i.e., the output of the pre-amplifier 212) and Vₓ is the intercept voltage. In accordance with an embodiment of the present invention, the logarithmic amplifier 216 has about 100 dB of dynamic range. An exemplary logarithmic amplifier 216 having such a dynamic range is the AD8310 98 dB Logarithmic Amplifier, available from Analog Devices of Norwood, Mass.

By compressing the dynamic range using the logarithmic amplifier 216, it is unnecessary to perform time gain correction (TGC) inside the probe 102 of the present invention. Rather, as mentioned above, and discussed in more detail below, the host computer 112 uses software to perform TGC. Additionally, because the logarithmic amplifier 216 performs envelope detection, the need to digitize radio frequency (RF) data is eliminated. This approach to ultrasound imaging also eliminates the need for electronic beamforming, which is required by an ultrasound imaging system that employs a transducer array.

The output of the logarithmic amplifier 216, which is a log-compressed and envelope-detected echo signal, is provided to an analog-to-digital converter (A/D) 218. The A/D 218 samples the log-compressed and envelope detected echo signal (e.g., at 30 or 48 MHz), to thereby digitize the signal. The A/D 216 is preferably an 8-bit analog-to-digital converter, because the cost of such a device is relatively inexpensive as compared to analog-to-digital converters with higher resolution. An exemplary A/D 216 is the ADC08L060 8-bit analog-to-digital converter available from National Semiconductor Corp. of Santa Clara, Calif. Nevertheless, analog-to-digital converters with other resolution are also within the scope of the present invention.

For ease of implementation, space savings and cost considerations, it is preferred that the logarithmic amplifier 216 performs both logarithmic compression and envelope detection. However, in another embodiment of the present invention, a logarithmic compressor and an envelope detector, which are separate components, can be used to perform these finctions.

The interface IC 204 outputs a clock signal 205 that has a frequency (e.g., 30 or 48 MHz) selected by the host computer 112 via software. The clock signal 205 is provided to the CPLD 206 and the A/D 218. Where the interface IC 204 is a CY7C8014A EZ-USB FX2LP™ USB Microcontroller, the clock signal is produced at the IFCLK output pin of the interface IC 204.

The interface IC 204 also outputs controls signals that are used to set the pulse frequency, down-sampling rate, and other parameters inside the CPLD 206. The CPLD 206 uses the clock signal 205 (e.g., 30 or 48 MHz) to produce the pulse control signals 207 that are provided to the pulser 208. The CPLD 206 implements the logic functions and counters that are used to provide outputs of the A/D 218 to the interface IC 204. The CPLD 206 also provides the pulse control signal 207 to the pulser 208. An exemplary IC that can be used to implement the CPLD 206 is the XCR3064XL CPLD available from Xilinx of San Jose, Calif. A Field Programmable Gate Array (FPGA) or custom IC can be used in place of the CPLD, if desired.

As mentioned above, the pulser 208 is preferably a bi-polar pulser. The high and low times of the bipolar pulses produced by the pulser 208 can be, e.g., 1, 2, 3, or 4 clock periods in length, resulting in single-cycle bipolar pulses that are 2, 4, 6, or 8 clock periods in total length. These pulse periods correspond to bipolar pulse “frequencies” of 15, 7.5, 5.0, or 3.75 MHz (when a 30 MHz clock is used) or 24, 12, 8.0, or 6.0 MHz (when a 48 MHz clock is used). While the above mentioned clock frequencies and pulse frequencies have been provided for example, other clock and pulse frequencies are also within the scope of the present invention.

In accordance with specific embodiments of the present invention, to support different imaging depths, down-sampling is done by the CPLD 206. For example, down-sampling by 1, 2, 3, and 4 can be supported for each sample rate, resulting in effective sample rates of 30, 15, 10, and 7.5 MHz (when the 30 MHz clock is used) and 48, 24, 16, and 12 MHz (when the 48 MHz clock is used). After down-sampling, the CPLD 206 writes the downsampled digitized data (e.g., 2048 bytes) into buffers inside the interface IC 204, or separate buffers (not shown). For 512×512 pixel images, 2048 samples per return echo corresponds to a 4× over-sampling rate as described in the Richard et al. article entitled “Real-Time Ultrasonic Scan Conversion via Linear Interpolation of Oversampled Vectors,” Ultrasound Imaging, Vol. 16, pp. 109-123, April 1994, which is incorporated herein by reference. Assuming the speed of sound in tissue is 1540 m/s, then 2048 samples taken at 7.5 MHz corresponds to a maximum imaging depth of 21 cm, while 2048 samples taken at 48 MHz corresponds to a minimum imaging depth of 3.3 cm. While embodiments of the present invention are not limited to the use of only these eight sample frequencies, this approach simplifies the implementation.

In accordance with an embodiment, the encoder 252 outputs an index signal 260 and a pulse signal 262. When imaging, a software routine running on the microprocessor of the interface IC 204 (or a separate microprocessor within the probe 102) implements a servo control loop by monitoring the index and pulse signals 260 and 262 from the encoder 252. The microprocessor of the interface IC 204 generates a pulse width modulated (PWM) control signal 238 that is used to drive a buck regulator 240 to produce the correct motor voltage signal 264 for the rotational speed desired. For example, if the motor 250 is running too slowly, the PWM signal 238 is used to increase the motor voltage produced by the buck regulator 240, and, conversely, if the motor 250 is running too fast, the PWM signal 238 is used to decrease the motor voltage. The software routine running on the microprocessor of the interface IC 204 can also determine the position of the switch 270 from such information.

In accordance with an embodiment, the index signal 260 produced by the encoder 252 is asserted once per rotation of the motor 250, and the pulse signal 262 is asserted multiple times per rotation (e.g., 512 times per rotation, or 256 times per left/right or right/left transducer sweep). The CPLD 206 monitors the pulse signal 262 and performs a data acquisition cycle each time a new position (i.e., angle) of the switch 270 is detected. For each pulse signal 262, the CPLD 206 signals the pulser 206 to produce a pulse at one of several different available pulse frequencies and then transfers data (e.g., 2048 bytes of data) from the A/D 218 to the high-speed data transfer buffers inside (or outside) the interface IC 204. This data acquisition process happens without intervention from the microprocessor of the interface IC 204 or the host 212. Once in the buffers, the data samples can be read over the passive interface cable 106 by the host computer 112. As mentioned above, in one embodiment, the switch 270 can have 256 different positions (i.e., angles), which can be represented by a single byte. Of course, more positions can be represented if more than 8 bits are used to represent the position. When the interface IC 204 sends the logarithmically compressed and envelope detected digital data to the host computer 112, such position data is sent therewith. Collectively, the logarithmically compressed and envelope detected digital data and the position data can be referred to as vector data, because the data includes both magnitude data and direction data.

In accordance with a preferred embodiment, the power for the motor 250 and all of the circuitry inside the probe 102 is received from the host computer 112 through the passive interface cable 106. For example, where the passive interface cable 106 is a USB 2.0 compliant cable, a peripheral device connected to the cable 106 is allowed to draw ½ Amp at a nominal 5V. Versions of this invention have been used to image at 10 frames/second (5 revolutions per second on the motor 250) that draw as little as ¼ Amp from a standard USB interface cable, which is equivalent to 1.25 W.

In accordance with an embodiment, a linear regulator IC 230 with integrated power switches and low quiescent current requirements designed for USB applications is used to produce a 3.3V digital supply 232, a 3.3V analog voltage supply 234, as well as a switched 5V supply 236 to switch the power to the encoder 256 on and off. The 3.3V digital supply 232 powers the interface IC 204, the CPLD 206, the SPROM 246, and the bus 228. The 3.3V analog supply powers the preamp 212, the logarithmic amplifier 214, and the A/D 218. In a suspend mode (e.g., a USB suspend mode), a “shut down” signal preferably turns off the 5V power 236 to the encoder 252 and the 3.3V analog supply 234, to thereby save power. A P-Channel Field Effect Transistor (PFET) is used to turn off power to the HVPS 220 when the system is in suspend mode or simply in frozen mode and not imaging. An exemplary IC that can be used for the linear regulator IC 230 is the TPS2148 3.3-V LDO and Dual Switch for USB Peripheral Power Management IC, available from Texas Instruments of Dallas, Tex.

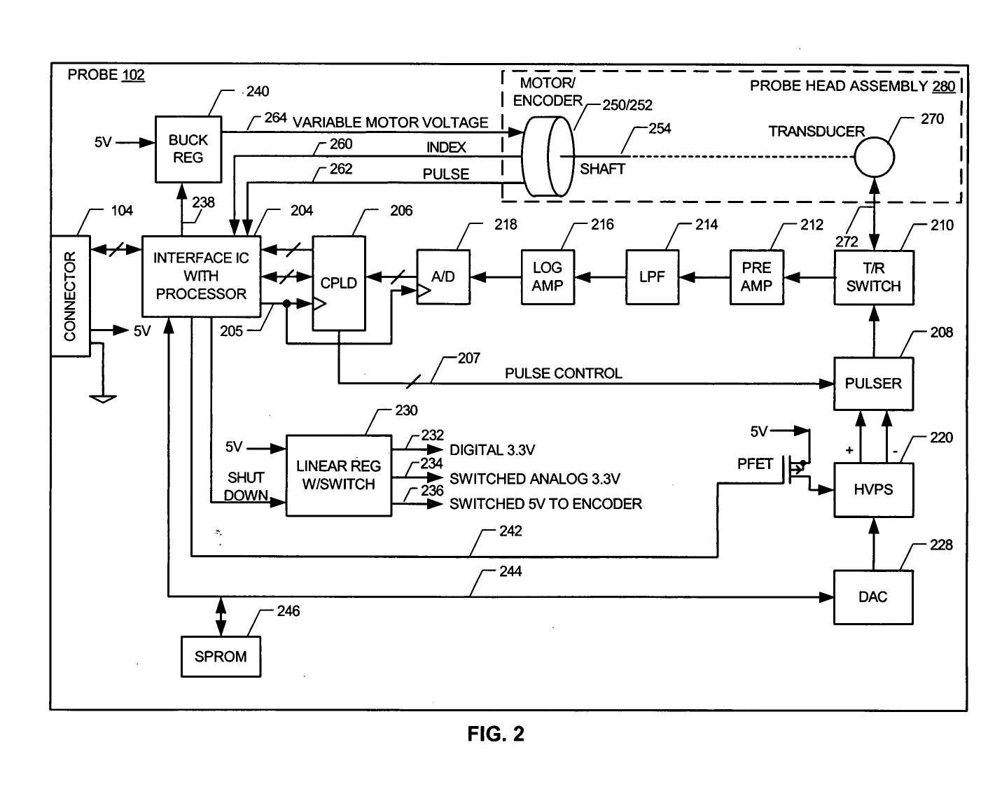

As mentioned above, the buck regulator 240 is used to produce the variable motor supply voltage 242 that drives the motor 250. FIG. 3 shows details of the buck regulator 240, according to an embodiment of the present invention. Power for the motor 250 comes from the passive interface cable 106 (e.g., a USB cable). When the probe 102 is not scanning, the PFET acts like an open switch. In this state, the PWM control voltage signal 238 from the interface IC 204 is in tri-state mode, and the PFET gate is pulled to 5V by the resistor R1. Pulling the PWM control signal 238 to ground turns the PFET on, i.e., closes the switch. By turning the PFET on and off using the PWM control signal 238 that alternates between the ground and tri-state drive levels, this standard buck regulator topology can produce any output voltage from 0V to the maximum voltage available from the interface cable 106 (e.g., nominally 5V). When the PFET is on (switch closed), current flows through an inductor L1 and charges a capacitor C1. When the PFET is off, the current through the inductor L1 continues to flow, at least briefly while the magnetic field collapses, and a diode D1 conducts. With proper sizing of the inductor L1 and the capacitor C1, and an appropriate PWM frequency, the circuit of FIG. 3 is employed to produce the variable voltage required by the motor 250 to run at the desired speed. Embodiments of the present invention also encompass the use of alternative regulator circuits.

FIG. 4 shows details of the HVPS 220, according to an embodiment of the present invention. In this embodiment, the HVPS 220 is a variable voltage, dual-rail high voltage power supply. As shown in FIG. 4, the HVPS 220 includes a charge pump control IC 402, a single-chip switched capacitor voltage doubler 404, an inductor L2, capacitors C2-C5, resistors R2-R5 and an N-channel field effect transistor NFET. An exemplary IC that can be used to provide the switched capacitor voltage doubler 404 is the LM2665 CMOS Switched Capacitor Voltage Converter available from National Semiconductor Corp. of Santa Clara, Calif. An exemplary IC that can be used to provide the charge pump control IC is the LM3478 High Efficiency Low-Side N-Channel Controller for Switching Regulator, also available from National Semiconductor Corp.

To provide an appropriate supply voltage for the charge pump control IC 402, the switched capacitor voltage double IC 404 is used to double the 5V supply voltage from the interface cable 106 (e.g., a USB cable) to approximately 10V. The charge pump inductor, L2, however, is fed directly from the 5V supply. The positive high voltage is generated in the standard manner. When the NFET closes, current builds up in the inductor L2. When the NFET opens, the current through the inductor L2 continues to flow, at least briefly, and the diode D2 conducts placing charge on the capacitor C2. By continuous “pumping,” the voltage on the capacitor C2 can go above the input voltage of 5V. The resistors R2 and R3 are used to feed back a portion of the output high voltage to the charge pump control IC 402, which turns the NFET on and off in a closed loop manner so that the desired high voltage is maintained. An exemplary IC that can be used to provide the NFET is IRF7494 Hexfet Power MOSFET available from International Rectifier of El Segundo, Calif.

In the standard charge pump topology, the resistor R4 is not used. Here, the output voltage from the bus 228 is used to inject current into the feedback circuit via the resistor R4. By controlling voltage output by the bus 228, the level of the output high voltage, shown here as +HV, can be controlled.

Two additional diodes, D3 and D4, and two additional capacitors, C3 and C4, are added to the standard charge pump DC-to-DC converter topology circuit to create the negative supply voltage, shown here as −HV. Generation of the −HV supply is similar to that described above for the +HV supply. The resistor R5 is chosen to be equal to the sum of the resistor R2 and R3 to provide a “bleeder” resistance from −HV to ground for safety purposes and to keep the circuit balanced. While −HV is not regulated directly, it will track the positive rail within a few percent in normal operation when the current drawn from the +HV and −HV power rails is approximately the same (as it is when a symmetric bipolar pulser is used). While FIG. 4, described above, provides details of the HVPS 220, according to an embodiment of the present invention. The use of alternative high voltage power supplies is also within the scope of the present invention.

The data samples produced by the ultrasound imaging probe 102 of the present invention are transmitted by the probe 102 across the interface cable 106 to the host computer 112. In a specific embodiment, this is accomplished when the host computer 112 reads the data temporarily stored in the buffers of the interface IC 204. The host computer 112 runs software that enables the host to perform time gain compensation (TGC), gray-scale mapping, and scan conversion of the data received from the probe 102, and the host displays the resultant video images. In the embodiment where the probe 102 includes only a single transducer, the host computer 112 does not need to perform electronic beamforming or other equivalent image processing, thereby simplifying the software that the host computer 112 runs.

The host computer 112 can use the digital data received from the ultrasound device 102 to provide any available type of ultrasound imaging mode can be used by the host computer 112 to display the ultrasound images, including, but not limited to A-mode, B-mode, M-mode, etc. For example, in B-mode, the host computer 112 performs know scan conversion such that the brightness of a pixel is based on the intensity of the echo return.

A benefit of specific embodiments of the present invention is that only digital signals are transmitted from the probe 102 to the host computer 112, thereby providing for better signal-to-noise ratio than if analog signals were transmitted from the probe 102 to the host computer 112, or to some intermediate apparatus between the host computer and the probe. Another benefit of specific embodiments of the present invention is that the switch 270 is in close proximity to (i.e., within the same housing as) the logarithmic amplifier 216 (or the separated logarithmic compressor and envelope detector) and the A/D 218. This will provide for good signal-to-noise (S/N) ratio, as compared to systems where the analog signals output by the switch 270 must travel across a relatively long distance before they are amplified and/or digitized. A further benefit of specific embodiments of the present invention is that the probe 102 does not perform any of electronic beamforming, time gain compensation, gray-scale mapping and scan conversion, thereby significantly decreasing the complexity, power requirements and cost of the probe 102. Another benefit of specific embodiments of the present invention is that the probe 102 can be used with a standard off-the-shelf passive interface cable.

Conventionally, finctions such as scan conversion, time gain correction (also known as time gain compensation) and gray-scale mapping are performed by a machine that is dedicated to obtaining ultrasound images, or by an intermediate device that is located between the probe and host computer. In contrast, here software running on the host computer 112 is used to perform these functions, thereby reducing the complexity and cost of the portable ultrasonic imaging probe 102.

The foregoing description of preferred embodiments of the present invention has been provided for the purposes of illustration and description. It is not intended to be exhaustive or to limit the invention to the precise forms disclosed. Many modifications and variations will be apparent to one of ordinary skill in the relevant arts. The above mentioned part numbers are exemplary, and are not meant to be limiting. Accordingly, other parts can be substituted for those mentioned above.

The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalence.

What is claimed is:

1. A portable ultrasonic imaging probe that is adapted to connect to a host computer via a passive interface cable, the portable ultrasound imaging probe comprising:
a probe head including a maneuverable single-element transducer to send ultrasonic pulses and detect ultrasonic echoes;
a logarithmic compressor to perform logarithmical compression of analog echo signals representative of the detected ultrasonic echoes;
an envelope detector to perform envelope detection of the logarithmically compressed analog echo signals;
an analog-to-digital converter to convert the logarithmically compressed and envelope detected analog echo signals to digital signals representative of the logarithmically compressed and envelope detected echo signals; and
interface circuitry to transfer the digital signals representative of the logarithmically compressed and envelope detected echo signals across a passive interface cable to a host computer that can perform time gain compensation, gray-scale mapping and scan conversion in order to display ultrasound images on a display associated with the host computer.

2. The portable ultrasonic imaging probe of claim 1, wherein a logarithmic amplifier comprises both the logarithmic compressor and the envelope detector, such that said logarithmic amplifier receives the analog echo signals representative of the detected ultrasonic echoes, performs both logarithmic compression and envelope detection of the analog echo signals, and outputs the logarithmically compressed and envelope detected analog echo signals.

3. The portable ultrasound imaging probe of claim 1, wherein the portable ultrasound imaging probe does not perform electronic beamforming.

4. The portable ultrasound imaging probe of claim 1, wherein the portable ultrasound imaging probe does not perform any of electronic beamforming, time gain compensation, gray-scale mapping and scan conversion.

5. The portable ultrasound imaging probe of claim 1, wherein said probe head assembly, said logarithmic compressor, said envelope detector, said analog-to-digital converter and said interface circuitry all receive power from the host computer via the same passive interface cable across which the probe transfers the digital signals to the host computer.

6. The portable ultrasound imaging probe of claim 5, further comprising voltage regulator circuitry to receive a power signal from the host computer via the passive interface cable, and to produce voltages used to power said probe head assembly, said logarithmic amplifier, said analog-to-digital converter and said interface circuitry.

7. The portable ultrasound imaging probe of claim 6, further comprising:
a pulser that provides high voltage pulses to said transducer to cause said transducer to send ultrasonic pulses; and
a high voltage power supply to step-up the voltage of the power signal, received from the host computer via the passive interface cable, to thereby produce a higher voltage that powers said pulser.

8. The portable ultrasound imaging probe of claim 1, further comprising:
a pre-amplifier; and
a filter;
wherein the analog echo signals are preamplified and filtered by said pre-amplifier and said filter before being provided to said logarithmic compressor.

9. The portable ultrasound imaging probe of claim 1, wherein said probe head assembly includes a motor to maneuver said transducer.

10. The portable ultrasound imaging probe of claim 1, wherein the passive interface cable via which the portable imaging probe is adapted to connect to the host computer is a standard USB 2.0 peripheral interface cable or a standard IEEE 1394 “Firewire” peripheral interface .

11. The portable ultrasound imaging probe of claim 1, wherein said probe head assembly, said logarithmic amplifier, said analog-to-digital converter and said interface circuitry all receive power from the host computer via a standard USB 2.0 peripheral interface cable or a standard IEEE 1394 “Firewire” peripheral interface cable that connects the portable ultrasound imaging probe to the host computer.

12. A method for providing efficient ultrasound imaging using a portable ultrasound imaging probe that is adapted connect to a host computer via a passive interface cable, method comprising:
(a) sending ultrasonic pulses using a transducer of the portable ultrasound imaging probe;
(b) detecting, at the transducer, ultrasonic echoes;
(c) performing, within the portable ultrasound imaging probe, logarithmic compression and envelope detection of analog echo signals representative of the detected ultrasonic echoes, to thereby produce logarithmically compressed and envelope detected analog echo signals;
(d) converting, within the portable ultrasound imaging probe, the logarithmically compressed and envelope detected analog echo signals to digital signals representative of the logarithmically compressed and envelope detected echo signals; and
(e) transferring the digital signals representative of the logarithmically compressed and envelope detected echo signals from the portable ultrasound imaging probe across a passive interface cable to a host computer that can perform time gain compensation, gray-scale mapping and scan conversion in order to display ultrasound images on a display associated with the host computer.

13. The method of claim 12, wherein electronic beamforming is not performed within the portable ultrasonic imaging probe.

14. The method of claim 12, wherein none of electronic beamforming, time gain compensation, gray-scale mapping and scan conversion are performed within the portable ultrasound imaging probe.

15. The method of claim 12, wherein none of electronic beamforming, time gain compensation, gray-scale mapping and scan conversion have been performed on the digital signals that are being transferred from the portable ultrasound imaging probe across the passive interface cable to the host computer.

16. The method of claim 12, further comprising:
receiving a power signal from the host computer via the passive interface cable; and
producing, from the power signal, voltages used to power components of the portable imaging probe that perform steps (a)-(e).

17. A portable ultrasound imaging probe that is adapted to be connected to a host computer via a passive interface cable, the portable ultrasound imaging probe comprising:
a maneuverable ultrasound transducer to send ultrasound signals and detect ultrasound echo signals;
a logarithmic amplifier to receive analog echo signals representative of the detected ultrasonic echoes, perform logarithmic compression and envelope detection of the analog echo signals, and output the logarithmically compressed and envelope detected analog echo signals;
an analog-to-digital converter to convert the logarithmically compressed and enveloped detected analog echo signals into digital signals; and
wherein the digital signals are transferred from the portable ultrasound imaging probe to a host computer via a passive interface cable.

18. The portable ultrasound imaging probe of claim 17, wherein the portable ultrasound imaging probe does not perform electronic beamforming.

19. The portable ultrasound imaging probe of claim 17, wherein the portable ultrasound imaging probe does not perform any of electronic beamforming, time gain compensation, gray-scale mapping and scan conversion.

20. The portable ultrasound imaging probe of claim 17, wherein said transducer, said logarithmic amplifier and said analog-to-digital converter all receive power from the host computer via the same passive interface cable across which the probe transfers the digital signals to the host computer.
